(19) United States
(12) Patent Application Publication
(10) Pub. No.: US 2020/0002767 A1
(43) Pub. Date: Jan. 2, 2020

(54) SQSTM1 MUTATIONS IN AMYOTROPHIC LATERAL SCLEROSIS

(71) Applicant: NORTHWESTERN UNIVERSITY, EVANSTON, IL (US)

(72) Inventors: Teepu Siddique, Wilmette, IL (US); Faisal Fecto, Chicago, IL (US)

(21) Appl. No.: 16/271,218

(22) Filed: Feb. 8, 2019

Related U.S. Application Data
(63) Continuation of application No. 15/470,185, filed on Mar. 27, 2017, now abandoned.
Application No. 15/470,185 is a division of application No. 13/588,870, filed on Aug. 17, 2012, now abandoned.
(60) Provisional application No. 61/525,548, filed on Aug. 19, 2011.

(51) Int. Cl. C12Q 1/6883; G01N 33/68

(52) U.S. Cl. CPC C12Q 1/6883; C12Q 2600/156; G01N 33/6896

(57) ABSTRACT

Provided herein is technology relating to diagnosing, monitoring, and treating disease and particularly, but not exclusively, to methods, compositions, and kits for diagnosing, monitoring, and treating amyotrophic lateral sclerosis by detecting and identifying mutations in the gene SQSTM1 and providing therapies by targeting aberrant biological functions related to mutant forms of SQSTM1.

This application is a continuation of U.S. patent application Ser. No. 15/470,185, filed Mar. 27, 2017, which is a divisional of U.S. patent application Ser. No. 13/588,870, filed Aug. 17, 2012, which claims priority to U.S. Prov. Pat. Appl. Ser. No. 61/525,548, filed Aug. 19, 2011, each of which is incorporated herein by reference in its entirety.

### STATEMENT REGARDING FEDERALLY SPONSORED RESEARCH OR DEVELOPMENT

This invention was made with government support under Grant NS050641 awarded by the National Institutes of Health. The government has certain rights in the invention.

### FIELD OF INVENTION

Provided herein is technology relating to diagnosing, monitoring, and treating disease and particularly, but not exclusively, to methods, compositions, and kits for diagnosing, monitoring, and treating amyotrophic lateral sclerosis by detecting and identifying mutations in the gene SQSTM1 and providing therapies by targeting aberrant biological functions related to mutant forms of SQSTM1.

### BACKGROUND

Amyotrophic lateral sclerosis (ALS) is a fatal paralytic disorder caused by degeneration of motor neurons in the brain and spinal cord. About 90% of ALS is sporadic (SALS) with unknown etiology. Familial ALS (FALS) is genetically heterogeneous and represents around 5 to 10% of ALS. The penetrance of genetic mutation-linked FALS may vary substantially, ranging from a classic Mendelian pattern to apparently sporadic disease. Mutations in the Cu/Zn superoxide dismutase gene (SOD1) represent the most prevalent known cause of ALS and account for approximately 20% of FALS and 1% of SALS cases (1, 2). Recently, several other genes, including TARDBP, FUS, OPTN, and VCP have been linked to ALS (3-9).

The SQSTM1 gene encodes the ubiquitin-binding protein p62 (also known as sequestosome (1)). p62 was initially identified as a novel ubiquitin-binding protein that acts as a phospho-tyrosine independent ligand of the p561ck SH2 domain (10, 11). Subsequently, p62 has been shown to have an important dual role in protein degradation both via the proteasome (12) and as a link between protein aggregation and autophagy (13) via its interaction with LC3/Atg8 (14). Dysfunction in these pathways has been shown to be implicated in various forms of neurodegeneration. p62 is present in neuronal and glial ubiquitin-positive inclusions of Alzheimer disease, Pick disease, dementia with Lewy bodies, Parkinson disease, and multiple system atrophy (15, 16). More recently, p62 was shown to aggregate in patients with ALS and the G93A SOD1 mouse model of FALS (17, 18). Overexpression of p62 with mutant SOD1 in NSC34 cells greatly enhances aggregate formation and this effect is significantly diminished when the ubiquitin-association domain (UBA) of p62 is deleted (17). Mutant SOD1 can be recognized by p62 in a ubiquitin-independent fashion and targeted for autophagy (19). p62 co-localizes with TDP-43 in brains of patients with frontotemporal lobe degeneration (FTLD) with motor neuron disease (20).

Recently, p62 was shown to co-localize with FUS and TDP-43 in ubiquitinated inclusions in motor neurons in spinal cords from patients with SALS, non-SOD1 FALS, and ALS with dementia (21). Recently, it has been shown that over-expression of p62 reduces TDP-43 aggregation in an autophagy and proteasome-dependent manner (22). p62 knockout mice develop memory loss subsequent to neurodegeneration caused by accumulation of hyperphosphorylated tau and neurofibrillary tangles (23). Recent studies have shown that though some proteins may participate in pathogenic aggregates in a wide variety of neurodegenerative disorders, they cause very specific disease phenotypes when mutant.

### SUMMARY

Provided herein is technology relating to diagnosing, monitoring, and treating disease and particularly, but not exclusively, to methods, compositions, and kits for diagnosing, monitoring, and treating ALS by detecting and identifying mutations in the gene SQSTM1 and providing therapies by targeting aberrant biological functions related to mutant forms of SQSTM1. Experiments described herein identified mutations in the SQSTM1 gene from ALS patients, thus providing diagnostic and therapeutic methods for managing ALS.

Accordingly, provided herein is technology related to methods for identifying a subject having amyotrophic lateral sclerosis (ALS) or predisposed to have ALS, the method comprising providing contacting a sample from the subject with a detection reagent adapted to detect a mutation in a SQSTM1 gene or a mutant SQSTM1 protein; and detecting, in vitro, a mutation in a SQSTM1 gene or a mutant SQSTM1 protein, wherein detecting a mutation in a SQSTM1 gene or a mutant SQSTM1 protein identifies the subject as having ALS or as predisposed to have ALS. In particular, in some embodiments of the technology, the mutation is detected in a nucleic acid, e.g., a DNA or an RNA. Embodiments provide for the analysis of a nucleic acid or a protein from a sample; that is, in some embodiments the sample comprises a biological molecule that is a genomic DNA, a mRNA, or a protein.

The technology is not limited in the technology used to query a nucleic acid or a protein for the presence of a mutation in a gene or a mutant protein. For example, in some embodiments a mutation in SQSTM1 is detected in an amplification product produced from a nucleic acid (e.g., using amplification reagents such as synthetic oligonucleotide primers and polymerase enzymes). In addition, some embodiments provide that the mutation is detected by nucleic acid sequencing, SNP detection, Southern blot, Northern blot, PCR, hybridization, restriction digest, nuclease mapping, electrophoresis, SSCP, or RT-PCR. Moreover, in some embodiments the mutation is detected in a protein (e.g., via use of antibodies or other ligand-specific binding partner), e.g., by Western blot, immunoassay, ELISA, electrophoresis, anti-phospho amino acid antibodies, protein sequencing, proteolysis, functional assay, structure determination, or by a measurement of a size or mass of a protein or protein fragment (e.g., by electrophoresis, mass spectrometry, column methods, etc.). Kits and components (e.g., detection reagents) employed in such techniques are described, for example, in U.S. Pat. Nos. 4,683,195, 4,683,202, 4,800,159, 4,965,188 5,480,784, 5,399,491, 5,824,518, 5,455,166, 5,130,238, 5,928,862. 5,283,174, 6,303,305, 6,541,205, 5,710,029, and 5,814,447; U.S. Publ. No. 20060046265 and 20050042638; and, Murakawa et al., DNA 7: 287 (1988), Weiss, R., Science 254: 1292 (1991), Walker, G. et al., Proc. Natl. Acad. Sci. USA 89: 392-396 (1992), Kwoh et al., Proc. Natl. Acad. Sci. USA 86:1173 (1989), and Guatelli et al., Proc. Natl. Acad. Sci. USA 87: 1874 (1990), each of which is herein incorporated by reference in its entirety.

The technology is not limited in the types of mutations in SQSTM1 that are detected. As such, the mutations may be missense, nonsense, deletion, insertion, intronic, silent, and/or splicing mutations. In some embodiments, particular mutations and/or mutant proteins are detected such as mutant SQSTM1 proteins having the following substitutions or deletions: A33V, V153I, P228L, V234V, K238del, H261H, S318P, R321C, S370P, P392L, G411S, and/or G425R, and/or SQSTM1 genes having the following mutations: c.98C>T, g.3′+7G>C, c.457G>A, g.5′-37C>T, c.683C>T, c.702G>A, c.714-716delGAA, c.783C>T, c.952T>C, c.961C>T, c.1108T>C, c.1175C>T, c.1231G>A, or c.1273G>A.

These mutations and mutant proteins result in various changes in protein structure and/or function that are relevant to the technology. For instance, in some embodiments, a mutation in SQSTM1 results in the production of a mutant protein comprising a change in a conserved region. In some embodiments, the mutation affects the phosphorylation of a protein. In some embodiments, the changes to the protein are in known domains. For example, in some embodiments, the mutation produces a mutant protein comprising a change in a Src homology domain (SH2); ZZ-type zinc finger domain; tumor necrosis factor receptor-associated factor 6 (TRAF6) binding site; a domain enriched in proline, glutamate, serine, and threonine (PEST domain); and/or a ubiquitin-association domain (UBA).

The technology relates to diagnosing and treating subjects with ALS. Accordingly in some embodiments of the technology the subject has or is predisposed to have familial amyotrophic lateral sclerosis and in some embodiments the subject has or is predisposed to have sporadic amyotrophic lateral sclerosis. In some embodiments, the subject suffers from or is predisposed to suffer from neurodegeneration and in some embodiments, the subject suffers from or is predisposed to suffer from Paget disease of bone (PDB).

Mutations in proteins such as SQSTM1 can lead to aberrant physiological and biological processes that, in turn, can produce disease symptoms. For example, in some embodiments, a mutation is associated with a biological abnormality in a biological process such as protein aggregation, protein folding, protein degradation, protein phosphorylation, and/or ubiquitination.

The technology provided herein finds use in manufacturing pharmaceuticals and other compositions related to treating or diagnosing ALS. For example, some embodiments provide for the use of a mutation in a SQSTM1 gene or a mutant SQSTM1 protein for the manufacture of a medicament to treat ALS. In some embodiments, molecules or other biologically active agents are designed by reference to a molecular model of a wild-type or mutant protein. For example, some embodiments provide for the use of a mutation in a SQSTM1 gene or a mutant SQSTM1 protein for the manufacture of a medicament to treat ALS by constructing a molecular model of the wild-type or mutant SQSTM1 protein and, e.g., designing agents that interact with the modeled protein.

In related embodiments, the technology provides for the use of a mutation in a SQSTM1 gene or a mutant SQSTM1 protein to develop a detection reagent. In some embodiments, the detection reagent is a biological molecule that is a nucleic acid or a protein, e.g., a probe, a primer, or an antibody.

Compositions are encompassed by the technology described. For example, some embodiments provide a composition comprising a first detection reagent adapted to detect a known marker of ALS and a second detection reagent adapted to detect a mutation in a SQSTM1 gene or a mutant SQSTM1 protein. Related embodiments provide a composition comprising a detection reagent hybridized or bound to a mutated SQSTM1 gene or a mutant SQSTM1 protein. In some embodiments of compositions, the detection reagent is a biological molecule that is a nucleic acid or a protein, e.g., a probe, a primer, or an antibody. In particular, some embodiments provide that the reagent is an antibody adapted to detect specifically a mutant SQSTM1 protein or an oligonucleotide adapted to detect specifically a mutation in a SQSTM1 gene. Particular embodiments are related to an antibody that detects a mutant SQSTM1 protein comprising a substitution or deletion that is A33V, V153I, P228L, V234V, K238del, H261H, S318P, R321C, S370P, P392L, G411S, and/or G425R. Moreover, some embodiments relate to an oligonucleotide probe or primer that specifically detects a mutation in the SQSTM1 gene that is c.98C>T, g.3′+7G>C, c.457G>A, g.5′-37C>T, c.683C>T, c.702G>A, c.714-716delGAA, c.783C>T, c.952T>C, c.961C>T, c.1108T>C, c.1175C>T, c.1231G>A, and/or c.1273G>A. Such embodiments of compositions find use in characterizing a biological sample.

In addition, such embodiments of compositions find use in kits, wherein they are packaged with an instruction for using the composition. Some embodiments relate to diagnostic systems comprising a functionality to analyze a sample, a functionality to detect a mutation in a SQSTM1 gene or a mutant SQSTM1 protein, and a functionality to report a result to a user indicating a risk of ALS. In some embodiments of the diagnostic systems, a sample is analyzed. The technology is not limited in the types of samples that are analyzed by the system, for example, the sample may comprise a nucleic acid or a protein. In some embodiments of the systems provided, the system comprises a functionality to analyze a sample, e.g., according to a method as provided herein and/or that is adapted to use a composition as provided herein (e.g., a detection reagent). Further embodiments of diagnostic systems comprise a functionality to analyze a sample, a functionality to detect a mutation in a SQSTM1 gene or a mutant SQSTM1 protein, a functionality to report a result to a user indicating a risk of ALS, and a detection reagent, e.g., as provided herein.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1A-FIG. 1C is a drawing of the SQSTM1 gene showing the positions of mutations associated with p62 pathology in ALS. FIG. 1A shows the structure of the SQSTM1 gene and indicates the coding regions (thick bars) and introns (thin lines). The location of each mutation identified in ALS is shown by diamonds above the exons. FIG. 1B shows the primary structure of p62 and indicates its major domains (SH2=Src homology 2 domain, AID=acidic interaction domain, ZZ=Zinc finger domain, TRAF6=tumor necrosis factor receptor-associated factor 6 binding domain, PEST=Proline, Glutamine, Serine, Threonine rich region, UBA=ubiquitin-associated domain). The arrows indicate the position of each amino acid change indentified in the cohort studied. FIG. 1C is an alignment of p62 protein sequences from different species. Sequence cluster alignment was done using HomoloGene (NCBI), which uses blastp to compare related sequences. Mutated residues are boxed. Sequences used include: NP_003891.1 (Homo sapiens), XP_518154.2 (Pan troglodytes), NP_788814.1 (Bos taurus), NP_035148.1 (Mus musculus), NP_787037.2 (Rattus norvegicus), XP_001233249.1 (Gallus gallus), and NP_998338.1 (Danio rerio).

### DETAILED DESCRIPTION

### Definitions

To facilitate an understanding of the present technology, a number of terms and phrases are defined below. Additional definitions are set forth throughout the detailed description.

As used herein, “a” or “an” or “the” can mean one or more than one. For example, “a” cell can mean one cell or a plurality of cells.

The term “patient” or “subject” refers to a human or other animal, such as a guinea pig or mouse and the like, capable of having a disease (e.g., ALS), either naturally occurring or induced.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “nascent” when used in reference to a protein refers to a newly synthesized protein, which has not been subject to post-translational modifications, which includes but is not limited to glycosylation and polypeptide shortening. The term “mature” when used in reference to a protein refers to a protein which has been subject to post-translational processing and/or which is in a cellular location (such as within a membrane or a multi-molecular complex) from which it can perform a particular function which it could not if it were not in the location.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or more amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure, or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The nomenclature used to describe variants of nucleic acids or proteins specifies the type of mutation and base or amino acid changes. For a nucleotide substitution (e.g., 76A>T), the number is the position of the nucleotide from the 5′ end, the first letter represents the wild type nucleotide, and the second letter represents the nucleotide which replaced the wild type. In the given example, the adenine at the 76th position was replaced by a thymine. If it becomes necessary to differentiate between mutations in genomic DNA, mitochondrial DNA, complementary DNA (cDNA), and RNA, a simple convention is used. For example, if the 100th base of a nucleotide sequence is mutated from G to C, then it would be written as g.100G>C if the mutation occurred in genomic DNA, m.100G>C if the mutation occurred in mitochondrial DNA, c.100G>C if the mutation occurred in cDNA, or r.100g>c if the mutation occurred in RNA. For amino acid substitution (e.g., D111E), the first letter is the one letter code of the wild type amino acid, the number is the position of the amino acid from the N-terminus, and the second letter is the one letter code of the amino acid present in the mutation. Nonsense mutations are represented with an X for the second amino acid (e.g. D111X). For amino acid deletions (e.g. ΔF508, F508del), the Greek letter Δ (delta) or the letters “del” indicate a deletion. The letter refers to the amino acid present in the wild type and the number is the position from the N terminus of the amino acid where it is present in the wild type. Intronic mutations are designated by the intron number or cDNA position and provide either a positive number starting from the G of the GT splice donor site or a negative number starting from the G of the AG splice acceptor site. g.3′+7G>C denotes the G to C substitution at nt+7 at the genomic DNA level. When the full-length genomic sequence is known, the mutation is best designated by the nucleotide number of the genomic reference sequence. See den Dunnen & Antonarakis, “Mutation nomenclature extensions and suggestions to describe complex mutations: a discussion”. Human Mutation 15: 7-12 (2000); Ogino S, et al., “Standard Mutation Nomenclature in Molecular Diagnostics: Practical and Educational Challenges”, J. Mol. Diagn. 9(1): 1-6 (February 2007), incorporated herein by reference in their entireties for all purposes.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

In particular, the term “SQSTM1 gene” refers to a full-length SQSTM1 nucleotide sequence. However, it is also intended that the term encompass fragments of the SQSTM1 sequence, as well as other domains with the full-length SQSTM1 nucleotide sequence. Furthermore, the terms “SQSTM1 nucleotide sequence” or “ SQSTM1 polynucleotide sequence” encompasses DNA, genomic DNA, cDNA, and RNA (e.g., mRNA) sequences.

The term “nucleotide sequence of interest” or “nucleic acid sequence of interest” refers to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “structural” when used in reference to a gene or to a nucleotide or nucleic acid sequence refers to a gene or a nucleotide or nucleic acid sequence whose ultimate expression product is a protein (such as an enzyme or a structural protein), an rRNA, an sRNA, a tRNA, etc.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. The terms encompass sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The term “polymorphic locus” refers to a genetic locus present in a population that shows variation between members of the population (i.e., the most common allele has a frequency of less than 0.95). Thus, “polymorphism” refers to the existence of a character in two or more variant forms in a population. A “single nucleotide polymorphism” (or SNP) refers a genetic locus of a single base which may be occupied by one of at least two different nucleotides. In contrast, a “monomorphic locus” refers to a genetic locus at which little or no variations are seen between members of the population (generally taken to be a locus at which the most common allele exceeds a frequency of 0.95 in the gene pool of the population).

A “frameshift mutation” refers to a mutation in a nucleotide sequence, usually resulting from insertion or deletion of a single nucleotide (or two or four nucleotides) which results in a change in the correct reading frame of a structural DNA sequence encoding a protein. The altered reading frame usually results in the translated amino-acid sequence being changed or truncated.

A “splice mutation” refers to any mutation that affects gene expression by affecting correct RNA splicing. Splicing mutation may be due to mutations at intron-exon boundaries which alter splice sites.

The term “detection assay” refers to an assay for detecting the presence or absence of a wild-type or variant nucleic acid sequence (e.g., mutation or polymorphism) in a given allele of a particular gene (e.g., SQSTM1 gene), or for detecting the presence or absence of a particular protein (e.g., SQSTM1) or the activity or effect of a particular protein or for detecting the presence or absence of a variant of a particular protein.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York [1989] pp. 16.7-16.8). A commonly used splice donor and acceptor site is the splice junction from the 16S RNA of SV40.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “response,” when used in reference to an assay, refers to the generation of a detectable signal (e.g., accumulation of reporter protein, increase in ion concentration, accumulation of a detectable chemical product).

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### Embodiments of the Technology

Provided herein is technology relating to diagnosing, monitoring, and treating disease and particularly, but not exclusively, to methods, compositions, and kits for diagnosing, monitoring, and treating amyotrophic lateral sclerosis by detecting and identifying mutations in the gene SQSTM1 and providing therapies by targeting aberrant biological functions related to mutant forms of SQSTM1.

Accordingly, provided herein are technologies relating to the association of mutations in the SQSTM1 gene and the incidence of familial and sporadic ALS cases. In particular, the data provided herein show that mutations in the SQSTM1 gene were identified in ˜2-3% of a large cohort of familial and sporadic ALS cases from unrelated families. This frequency is similar to what has been reported for other genes involved in ALS, namely, FUS, TARDBP, VCP and ANG (9). While an understanding of the mechanism is not required to practice the technology and the technology is not bound by any particular theory, the SQSTM1 mutations may be associated with several modes of biological action. For example, the SQSTM1 mutations may confer a toxic gain of function through novel protein interactions and subsequent deregulation of cell signaling pathways and/or the SQSTM1 mutations may lead to protein misfolding and aggregation. Moreover, the SQSTM1 mutations described herein may have low penetrance because most were present in individuals with a small pedigree structure of familial aggregates or sporadic cases, and not in large families. Other genes implicated in ALS like the PONs and ANG may also cause disease by low penetrance mutations because they have also been described in SALS and familial aggregates rather than large multigenerational pedigrees. Some low penetrance mutations in SOD1, FUS, and TARDBP have also been reported in apparent SALS. Since the approaches described were based on candidate gene sequencing as opposed to linkage analysis, the possibility exists that the identified changes represent rare, possibly functional, variants conferring increased risk rather than pathogenic mutations. One criterion suggesting that a group of rare variants in a certain gene influences inherited susceptibility is that they are over-represented in disease versus control groups (30). The data comprise 248 statistically significant differences between ALS and controls whether all exclusive variants, only missense/deletion variants, or only functionally relevant variants in the SQSTM1 gene were considered.

The data demonstrate that the variants found in the ALS cohort are pathogenic. Firstly, none of the variants present in the ALS cohort were detected in more than 724 controls (representing 1448 chromosomes), the dbSNP, and 1000 Genomes databases. Secondly, all these variants affect amino acids which show a high level of evolutionary conservation. Thirdly, in silico analysis predicts that nearly all of these variants have a deleterious effect on the structure and function of p62.

Histopathological studies have shown that p62 is present in ubiquitinated inclusions of both SOD1-positive FALS and other forms of ALS (17, 21), suggesting a common pathogenic mechanism. The data provided herein present a parallel between p62 and other proteins linked to neurodegeneration like TDP-43, FUS, optineurin, β-amyloid, α-synuclein, and tau. These proteins may aggregate in a wide variety of neurological disorders, but mutations in their genes cause very specific phenotypes in rare families. Such rare but pathogenic mutations provide a novel approach where the gene and its product can be investigated in molecular pathways at epigenetic, genetic, and post-translational levels for relevance to sporadic disease.

Genes linked to two distinct clinical syndromes are well known. For instance, mutations in TRPV4 that were previously linked to bony dysplasias were recently linked to axonal neuropathies (34). Moreover, mutations in the gene encoding valosin-containing protein (VCP) have been implicated to cause human neurodegeneration in the syndrome of inclusion body myopathy with Paget disease of bone (PDB) and/or FTLD (IBMPFD) (35). Recently, VCP mutations were described by Johnson et al. in ALS patients (8). Interestingly, one of the mutations (R191Q) described by Johnson et al. in their ALS cohort had already been described in IBMPFD families, and two other mutations from the same study (R159G and R155H) involved codons that had been found to be mutated in IBMPFD, highlighting the ability of the same mutation to confer variable clinical phenotypes.

Furthermore, optineurin, mutations in which were recently linked to ALS (7), was identified as a genetic risk factor for PDB in a recent genome-wide association study (36). The three UBA-domain mutations described in the ALS cohort examined herein have been previously identified in familial and sporadic PDB (37). This is intriguing because the co-existence of PDB and ALS, although not widely recognized, has been previously reported, hence suggesting a possible common link between these diseases (38). It is possible that this co-existence is under-reported because PDB, like ALS, is rarely diagnosed before 40 years of age, when symptoms of ALS being more severe and lethal would preclude PDB diagnosis. The data do not provide evidence of a family or personal history of PDB in the cohort and these mutations were absent in our control population. It has been reported that affected individuals from the same PDB family have variable and sometimes no expressivity of the disease even with one mutated copy of the SQSTM1 gene (39). Moreover, reported transgenic mouse models of PDB do not develop bone disease (40). This suggests that specific environmental factors or other modifier loci in addition to SQSTM1 mutations may be important in determining the specificity of the disease phenotype. Accordingly, in some embodiments of the technology a subject having amyotrophic lateral sclerosis (ALS) or predisposed to have ALS does not have Paget's disease of bone (PDB).

The data provided herein widen the clinical spectrum associated with SQSTM1 mutations to include ALS and show that some ALS patients should be monitored for features of PDB, and more broadly, altered bone metabolism. Some ALS patients, however, do not have PDB. It is contemplated that mutations in the SQSTM1 gene act to cause ALS either directly or through a modifier effect involving additional genes and/or environmental factors. The specific effects of these mutations in SQSTM1 on protein degradation pathways find use in identifying therapeutics for treating ALS and monitoring therapy.

Although the disclosure herein refers to certain illustrated embodiments, it is to be understood that these embodiments are presented by way of example and not by way of limitation.

### EXAMPLES

### Example 1

Participants. A cohort of 340 FALS and 206 SALS and 738 neurologically normal control subjects were ascertained from our Neurologic Diseases Registry, in which subjects are enrolled after informed consent is obtained. Pedigrees and clinical data were collected according to protocols approved by our Institutional Review Board and met Health Insurance Portability and Accountability Act standards of confidentiality and disclosure. All patients were diagnosed by board-certified neurologists and met the revised Escorial criteria for diagnosis of clinically definite, probable, or laboratory-supported probable ALS (24). All cases were negative for mutations in the SOD1, TARDBP, and FUS genes. By self-reported ethnicity 93.9% of the cases were white (European-American), 2.5% Asian, 1.9% African-American, and 1.3% Latino. The ethnicity of 2 cases was unknown. By self-reporting, 97.6% of controls were white, 0.56% African-American, 0.98% Latino, and 0.84% Asian.
Sequencing analysis of the SQSTM1 gene. Genomic DNA was extracted from transformed lymphoblastoid cell lines or whole blood using standard protocols (Qiagen, Valencia, Calif. Intronic primers covering the coding sequence were designed at least 50 bp away from the intron/exon boundaries. Primers were designed using Oligo Analyzer (IDT, Coralville, Iowa), ExonPrimer (Institute of Human Genetics, Germany), and UCSC Genome Browser. Genomic DNA was amplified according to standard protocols. Unconsumed dNTPs and primers were digested with Exonuclease I and Shrimp Alkaline Phosphatase (ExoSAP-IT, USB, Cleveland, Ohio). Fluorescent dye labeled single-stranded DNA was amplified with Beckman Coulter sequencing reagents (GenomeLab DTCS Quick Start Kit) followed by single pass bi-directional sequencing with CEQ™ 8000 Genetic Analysis System (Beckman Coulter, Fullerton, Calif.). Forward primer was used for mutation screening and all variations were confirmed by reverse sequencing. When a variant was identified, it was first excluded in the dbSNP and 1000 Genomes databases, and then a large number of normal control DNA samples (n>724; Table 1) were analyzed to exclude the possibility of a common polymorphism.

TABLE 1
SQSTM1 variants in familial and sporadic ALS

| | | | | | SALS + | |
|---|---|---|---|---|---|---|
| Exon | Change (bp) | Variant | FALS | SALS | FALS | Controls |
| 1 | c.98C > T | A33V | 1/340 | 2/206 | 3/546 | 0/724 |
| 1 | g.3’ + 7G > C | Intronic | 1/340 | 0/206 | 1/546 | 0/724 |
| 3 | c.457G > A | V153I | 0/340 | 2/206 | 2/546 | 0/724 |
| 4 | g.5’ − 37C > T | Intronic | 1/340 | 2/206 | 3/546 | 0/738 |
| 5 | c.683C > T | P228L | 0/340 | 1/206 | 1/546 | 0/724 |
| 5 | c.702G > A | V234V | 1/340 | 0/206 | 1/546 | 0/724 |
| 5 | c.714 − | K238del | 0/340 | 1/206 | 1/546 | 0/724 |
| | 716delGAA | | | | | |
| 6 | c.783C > T | H261H | 0/340 | 1/206 | 1/546 | 0/738 |
| 6 | c.952T > C | S318P | 1/340 | 0/206 | 1/546 | 0/738 |
| 6 | c.961C > T | R321C | 0/340 | 1/206 | 1/546 | 0/738 |
| 7 | c.1108T > C | S370P | 1/340 | 0/206 | 1/546 | 0/733 |
| 8 | c.1175C > T | P392L | 2/340 | 1/206 | 3/546 | 0/737 |
| 8 | c.1231G > A | G411S | 1/340 | 0/206 | 1/546 | 0/737 |
| 8 | c.1273G > A | G425R | 0/340 | 1/206 | 1/546 | 0/737 |

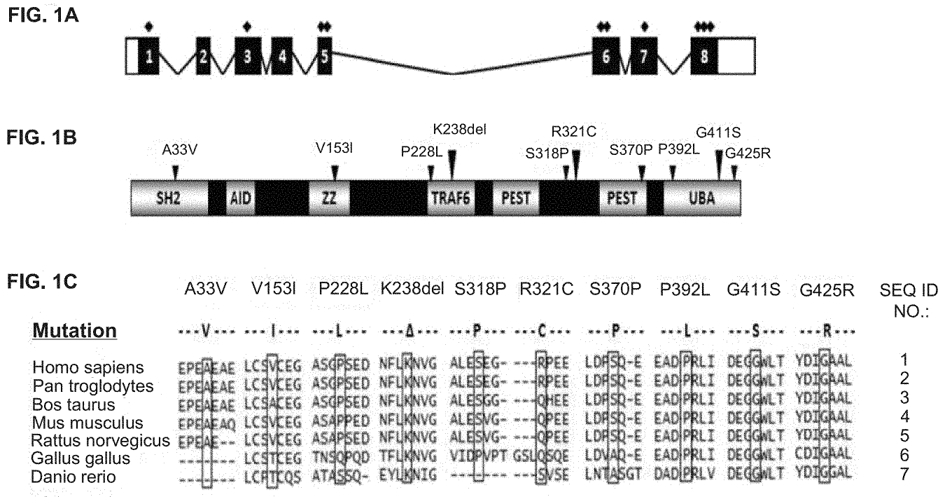

Bioinformatics. The NetPhos2.0 program was used to predict changes in phosphorylation sites in variants identified during sequencing (26). Variants were also analyzed using SIFT (27) and Pmut (28) programs to predict the effect of the mutations on p62. 3D-modeling was done with the Swiss-PdBViewer using 1Q02A backbone as a template (29).
Statistical Analysis. Data were analyzed using the PSPP for Windows. Case-control genotype associations were assessed by x² analyses and odds ratios were calculated. Estimations of departures from Hardy-Weinberg equilibrium (HWE) were calculated by x² test. Graph Pad 130 QuickCalcs was used to perform two-tailed Fisher exact test for comparing rare variant frequencies. Clinical data were analyzed using Graph Pad QuickCalcs and Kaplan-Meier analysis was performed using EpiInfo.
Results. The SQSTM1 gene is located on chromosome 5q35 and all of its eight exons are coding (FIG. 1A). To identify DNA mutations that predispose a subject to ALS, the entire coding region of SQSTM1 was sequenced in a cohort of 546 ALS patients (340 FALS and 206 SALS subjects and a total of 1092 chromosomes). Ten mutations (nine heterozygous missense and one deletion) were identified in 15 individuals of whom six had FALS and nine had SALS (Tables 1 and 2). None of these changes was present in more than 724 controls (representing 1448 chromosomes), the dbSNP and 1000 Genomes databases (Table 1). There was a personal history of Parkinsonism in two individuals. The A33V mutation was present in one case of FALS and two cases with SALS. The P392L substitution was found in two FALS probands but segregation analysis was not possible due to a lack of samples from additional family members. The P392L variant was also present in one individual with SALS. The V153I change was present in two SALS patients. The frequency of these variants in our cohort of ALS patients was 2.75%.

TABLE 2
Characterizaition of FALS probands & SALS patients with SQSTM1 mutations.

| Pedigree | Type | Amino acid change | Ethnicity | Gender | Age at onset (years) | Site | Duration (months) |
|---|---|---|---|---|---|---|---|
| 9436 | FALS | A33V | Hispanic | Male | 47 | Limb | 15 |
| 8588 | SALS | A33V | White | Female | 69 | Limb | 94 |
| 8253 | SALS | A33V | White | Male | 62 | Bulbar | alive at 85 |
| 1216 | SALS | V153I | White | Male | 55 | Limb | 25 |
| 8655 | SALS | V153I | White | Male | 65 | Limb | 65 |
| 8913 | SALS | P228L | White | Male | 33 | Limb | 51 |
| 8187 | SALS | K238del | White | Male | 57 | Limb | 20 |
| 954 | FALS | S318P | White | Female | 61 | Bulbar | 218 |
| 8105 | SALS | R321C | White | Female | 55 | Bulbar | 5 |
| 7165 | FALS | S370P | Af. Am/ | Male | 43 | Limb | alive at |
| | | | White | | | | 145 |
| 1318 | FALS | P392L | n/a | Male | n/a | n/a | n/a |
| 9064 | FALS/P | P392L | White | Female | 72 | Bulbar | 29 |
| 8257 | SALS | P392L | White | Female | 54 | Limb | 81 |
| 8516 | FALS | G411S | White | Male | 45 | Limb | 168 |
| 8796 | SALS/P | G425R | White | Male | 47 | Limb | 56 |

(FALS = familial ALS,
SALS = sporadic ALS,
P = Parkinsonism,
n/a = not available)

The data identified two silent and two intronic variants that were present exclusively in the tested ALS cohort and that were not present in the controls (Table 1). Several other rare and common variants were identified in both cases and controls and/or reported in dbSNP. Rare variants were defined as variations having frequencies less than 1% (30). A few rare variants were observed exclusively in controls. A statistically significant difference was observed when the frequency of all rare variants exclusively present in individuals with ALS was compared with the frequency of all rare variants exclusively found in controls (22/1092 versus 11/1448; p=0.0073; two-tailed Fisher exact test; Table 3). Moreover, a statistically significant difference was also observed when the frequency of only rare missense and/or deletion variants exclusively present in our cohort of ALS patients was compared with the frequency of such rare variants exclusively found in controls (16/1092 versus 9/1448; p=0.0414; two-tailed Fisher exact test; Table 3).

TABLE 3
Rare variant frequencies in individuals with ALS and controls

| | All rare variants | | Rare missense/deletion variants | |
|---|---|---|---|---|
| | ALS | Controls | ALS | Controls |
| Total Alleles | 1092 | 1448 | 1092 | 1448 |
| All variants | 31 | 26 | 21 | 14 |
| P-value | 0.1036 | | 0.0571 | |
| Exclusive variants | 22 | 11 | 16 | 9 |

| | | |
|---|---|---|
| P-value | 0.0073* | 0.0414* |

| | | |
|---|---|---|
| Exclusive functional variants | 13 | 6 |

| | |
|---|---|
| P-value | 0.0342* |

p62 is highly conserved in mammals (FIG. 1C). All the mutations identified in the ALS cohort were located in conserved regions of the p62 protein. Four of the 10 mutations observed in the 168 ALS cohort were fully conserved across seven species examined. Four mutated residues were conserved in five species. The Val153 residue was conserved in four species. Eight of the nine missense variants were predicted to have a harmful effect on the structure and function of p62 by at least one of two protein conformation prediction programs used. The frequency of functionally relevant rare variants exclusively present in individuals with ALS was significantly higher than the frequency of rare functional variants exclusively found in controls 174 (13/1092 versus 6/1448; p=0.0342; two-tailed Fisher exact test; Table 3).

The A33V substitution occurs in the Src homology 2 domain (SH2) (FIG. 1B). These domains are generally about 100 residues in length and are known to associate with phosphorylated tyrosine residues. The A33V change may affect phospho-tyrosine ligand binding and specificity, which may lead to altered function of p62 in protein tyrosine kinase pathways. Indeed, a number of mutations in SH2 domain proteins are already associated with several human diseases (31). The V153I mutation occurs in the ZZ-type zinc finger domain which is thought to be involved in protein-protein interactions and is present in proteins such as dystrophin. The P228L and K238del (deletion of the lysine at position 238) mutations occur in the binding site for the tumor necrosis factor receptor-associated factor 6 (TRAF6). The S318P and R321C mutations are not present in any known domains and may lead to abnormal protein folding and aggregation of p62. The S370P variant occurs in a PEST domain that is a region enriched in proline (P), glutamate (E), serine (5), and threonine 188 (T) residues, and phosphorylation in this domain marks proteins for proteolysis (32). Moreover, the Ser318 residue occurs between two PEST domains and may remove a crucial phosphorylation site and make the p62 protein more prone to aggregation. In fact, four of the 10 mutations seen in the ALS cohort were predicted to have an effect on p62 phosphorylation. Particularly, the S318P and S370P substitutions remove serine residues that are predicted to be highly probable phosphorylation sites. The UBA-domain of p62 forms a compact three-helix bundle. The P392L and G411S substitutions are present just outside the hydrophobic patch of helix-1 and helix-2, whereas the G425R change occurs within the hydrophobic patch of helix-3. These UBA-domain mutations may affect binding of p62 to ubiquitin or ubiquitinated proteins and may lead to accumulation of the ubiquitin-positive protein aggregates that are characteristic of ALS.

The clinical data obtained from 14 patients with SQSTM1 mutations were compared to the cohort of SOD1, TARDBP, and FUS mutant patients (33). The average age at symptom onset for patients with SQSTM1 mutations (54.6±10.9 years; n=14) was similar to those with TARDBP mutations (54.7±15.3 years; n=34), but later than those with FUS (43.6±15.8 years; 204 n=54; p=0.0169, two-tailed Student t-test) or SOD1 mutations (47.7±13.0 years; n=164; 205 p=0.05, two-tailed Student t-test). The association between age of onset and different ALS-linked genes was tested by comparing Kaplan-Meier survival curves and then evaluating the homogeneity of the survival curves by using the log-rank test and Wilcoxon test. No significant differences were observed with the log-rank test when we compared SQSTM1 mutant patients to SOD1, FUS, or TARDBP mutant patients. However, differences between SQSTM1 and FUS mutant patients were significant with the Wilcoxon test (p=0.0129), which is more sensitive than the log-rank test to differences between groups that occur at earlier time points. The average duration of symptoms was longer for patients with SQSTM1 mutations at 6.3±5.3 years (n=14) when compared to patients with FUS (3.4±5.7 years; n=44), SOD1 (4.1±4.9 years; n=144), and TARDBP mutations (3.3±2.3 years; n=3 0). The average duration of symptoms in patients with SQSTM1 mutations was nearly twice as long as those with TARDBP mutations (p=0.0114, two-tailed Student t-test). The duration of symptoms varied widely. However, 64% of SQSTM1 mutant patients survived beyond four years which was remarkably higher than patients with FUS (11.4%), SOD1 (29.9%), and TARDBP (30%) mutations. When comparing the site of symptom onset, we observed that SQSTM1 patients had a similar proportion of bulbar-onset (28.6%) compared to FUS (33.3%) and TARDBP (32.1%) but markedly higher than patients with SOD1 mutations (7.6%; p=0.05, two-tailed Fisher exact test).

### REFERENCES

1. Deng H X, Hentati A, Tainer J A, et al. Amyotrophic lateral sclerosis and structural defects in Cu,Zn superoxide dismutase. Science. Aug. 20, 1993; 261(5124):1047-1051.

2. Rosen D R, Siddique T, Patterson D, et al. Mutations in Cu/Zn superoxide dismutase gene are associated with familial amyotrophic lateral sclerosis. Nature. Mar. 4, 1993; 362(6415):59-62.

3. Kwiatkowski T J, Jr., Bosco D A, Leclerc A L, et al. Mutations in the FUS/TLS gene on chromosome 16 cause familial amyotrophic lateral sclerosis. Science. Feb. 27, 2009; 323(5918):1205-1208.

4. Vance C, Rogelj B, Hortobagyi T, et al. Mutations in FUS, an RNA processing protein, cause familial amyotrophic lateral sclerosis type 6. Science. Feb. 27, 2009; 323(5918):1208-1211.

5. Kabashi E, Valdmanis P N, Dion P, et al. TARDBP mutations in individuals with sporadic and familial amyotrophic lateral sclerosis. Nat Genet. May 2008; 40(5):572-574.

6. Sreedharan J, Blair I P, Tripathi V B, et al. TDP-43 mutations in familial and sporadic amyotrophic lateral sclerosis. Science. Mar. 21, 2008; 319(5870):1668-1672.

7. Maruyama H, Morino H, Ito H, et al. Mutations of optineurin in amyotrophic lateral sclerosis. Nature. May 13, 2010; 465(7295):223-226.

8. Johnson J O, Mandrioli J, Benatar M, et al. Exome sequencing reveals VCP mutations as a cause of familial ALS. Neuron. Dec. 9, 2010; 68(5):857-864.

9. Ticozzi N, Tiloca C, Morelli C, et al. Genetics of familial amyotrophic lateral sclerosis. Arch Ital Biol. March 2011; 149(1):65-82.

10. Joung I, Strominger J L, Shin J. Molecular cloning of a phosphotyrosine-independent ligand of the p561ck SH2 domain. Proc Natl Acad Sci USA. Jun. 11, 1996; 93(12):5991-348 5995.

11. Vadlamudi R K, Joung I, Strominger J L, Shin J. p62, a phosphotyrosine-independent ligand of the SH2 domain of p561ck, belongs to a new class of ubiquitin-binding proteins. J Biol Chem. Aug. 23, 1996; 271(34):20235-20237.

12. Seibenhener M L, Babu J R, Geetha T, Wong H C, Krishna N R, Wooten M W. Sequestosome 1/p62 is a polyubiquitin chain binding protein involved in ubiquitin proteasome degradation. Mol Cell Biol. September 2004; 24(18):8055-8068.

13. Bjorkoy G, Lamark T, Johansen T. p62/SQSTM1: a missing link between protein aggregates and the autophagy machinery. Autophagy. April-June 2006; 2(2):138-139.

14. Pankiv S, Clausen T H, Lamark T, et al. p62/SQSTM1 binds directly to Atg8/LC3 to facilitate degradation of ubiquitinated protein aggregates by autophagy. J Biol Chem. Aug. 17, 2007; 282(33):24131-24145.

15. Kuusisto E, Salminen A, Alafuzoff I. Ubiquitin-binding protein p62 is present in neuronal and glial inclusions in human tauopathies and synucleinopathies. Neuroreport. Jul. 20, 2001; 12(10):2085-2090.

16. Zatloukal K, Stumptner C, Fuchsbichler A, et al. p62 is a common component of cytoplasmic inclusions in protein aggregation diseases. Am J Pathol. January 2002; 160(1):255-263.

17. Gal J, Strom A L, Kilty R, Zhang F, Zhu H. p62 accumulates and enhances aggregate formation in model systems of familial amyotrophic lateral sclerosis. J Biol Chem. Apr. 13 2007; 282(15):11068-11077.

18. Mizuno Y, Amari M, Takatama M, Aizawa H, Mihara B, Okamoto K. Immunoreactivities of p62, an ubiqutin-binding protein, in the spinal anterior horn cells of patients with amyotrophic lateral sclerosis. J Neurol Sci. Nov. 1, 2006; 249(1):13-18.

19. Gal J, Strom A L, Kwinter D M, et al. Sequestosome 1/p62 links familial ALS mutant SOD1 to LC3 via an ubiquitin-independent mechanism. J Neurochem. November 2009; 111(4):1062-1073.

20. Hiji M, Takahashi T, Fukuba H, Yamashita H, Kohriyama T, Matsumoto M. White matter lesions in the brain with frontotemporal lobar degeneration with motor neuron disease: TDP-43-immunopositive inclusions co-localize with p62, but not ubiquitin. Acta Neuropathol. August 2008; 116(2):183-191.

21. Deng H X, Zhai H, Bigio E H, et al. FUS-immunoreactive inclusions are a common feature in sporadic and non-SOD1 familial amyotrophic lateral sclerosis. Ann Neurol. June 2010; 67(6):739-748.

22. Brady O A, Meng P, Zheng Y, Mao Y, Hu F. Regulation of TDP-43 aggregation by phosphorylation and p62/SQSTM1. J Neurochem. January 2011; 116(2):248-259.

23. Ramesh Babu J, Lamar Seibenhener M, Peng J, et al. Genetic inactivation of p62 leads to accumulation of hyperphosphorylated tau and neurodegeneration. J Neurochem. July 2008; 106(1):107-120.

24. Brooks B R. El Escorial World Federation of Neurology criteria for the diagnosis of amyotrophic lateral sclerosis. Subcommittee on Motor Neuron Diseases/Amyotrophic Lateral Sclerosis of the World Federation of Neurology Research Group on Neuromuscular Diseases and the El Escorial “Clinical limits of amyotrophic lateral sclerosis” workshop contributors. J Neurol Sci. July 1994; 124 Suppl:96-107.

25. Durbin R M, Abecasis G R, Altshuler D L, et al. A map of human genome variation from population-scale sequencing. Nature. Oct. 28, 2010; 467(7319):1061-1073.

26. Blom N, Gammeltoft S, Brunak S. Sequence and structure-based prediction of eukaryotic protein phosphorylation sites. J Mol Biol. Dec. 17, 1999; 294(5):1351-1362.

27. Ng P C, Henikoff S. SIFT: Predicting amino acid changes that affect protein function. Nucleic Acids Res. Jul. 1, 2003; 31(13):3812-3814.

28. Ferrer-Costa C, Gelpi J L, Zamakola L, Parraga I, de la Cruz X, Orozco M. PMUT: a web-based tool for the annotation of pathological mutations on proteins. Bioinformatics. Jul. 15, 2005; 21(14):3176-3178.

29. Guex N, Peitsch M C. SWISS-MODEL and the Swiss-Pdb Viewer: an environment for comparative protein modeling. Electrophoresis. December 1997; 18(15):2714-2723.

30. Bodmer W, Bonilla C. Common and rare variants in multifactorial susceptibility to common diseases. Nat Genet. June 2008; 40(6):695-701.

31. Lappalainen I, Thusberg J, Shen B, Vihinen M. Genome wide analysis of pathogenic SH2 domain mutations. Proteins. August 2008; 72(2):779-792.

32. Rogers S, Wells R, Rechsteiner M. Amino acid sequences common to rapidly degraded proteins: the PEST hypothesis. Science. Oct. 17, 1986; 234(4774):364-368.

33. Yan J, Deng H X, Siddique N, et al. Frameshift and novel mutations in FUS in familial amyotrophic lateral sclerosis and ALS/dementia. Neurology. Aug. 31, 2010; 75(9):807-814.

34. Deng H X, Klein C J, Yan J, et al. Scapuloperoneal spinal muscular atrophy and CMT2C are allelic disorders caused by alterations in TRPV4. Nat Genet. February 2010; 42(2):165-169.

35. Weihl C C, Pestronk A, Kimonis V E. Valosin-containing protein disease: inclusion body myopathy with Paget's disease of the bone and fronto-temporal dementia. Neuromuscul Disord. May 2009; 19(5):308-315.

36. Albagha O M, Visconti M R, Alonso N, et al. Genome-wide association study identifies variants at CSF1, OPTN and TNFRSF11A as genetic risk factors for Paget's disease of bone. Nat Genet. June 2010; 42(6):520-524.

37. Michou L, Collet C, Laplanche J L, Orcel P, Cornelis F. Genetics of Paget's disease of bone. Joint Bone Spine. May 2006; 73(3):243-248.

38. Varelas P N, Bertorini T E, Kapaki E, Papageorgiou C T. Paget's disease of bone and motor neuron disease. Muscle Nerve. May 1997; 20(5):630.

39. Leach R J, Singer F R, Ench Y, Wisdom J H, Pina D S, Johnson-Pais T L. Clinical and cellular phenotypes associated with sequestosome 1 (SQSTM1) mutations. J Bone Miner Res. December 2006; 21 Suppl 2:P45-50.

40. Kurihara N, Hiruma Y, Zhou H, et al. Mutation of the sequestosome 1 (p62) gene increases osteoclastogenesis but does not induce Paget disease. J Clin Invest. January 2007; 117(1):133-142.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

We claim:

1. A method for identifying a subject having amyotrophic lateral sclerosis (ALS) or predisposed to have ALS, the method comprising
a) contacting a sample from the subject with a detection reagent adapted to detect:
1) a mutation in a SQSTM1 gene; or
2) a mutant SQSTM1 protein; and
b) detecting, in vitro:
1) a mutation in a SQSTM1 gene; or
2) a mutant SQSTM1 protein,
wherein detecting a mutation in a SQSTM1 gene or a mutant SQSTM1 protein identifies the subject as having ALS or as predisposed to have ALS.

2. The method of claim 1 wherein the mutation is detected in a nucleic acid.

3. The method of claim 1 wherein the mutation is detected in an amplification product produced from a nucleic acid.

4. The method of claim 1 wherein the mutation is detected by a method selected from the group consisting of nucleic acid sequencing, SNP detection, Southern blot, Northern blot, PCR, hybridization, invader assay, restriction digest, nuclease mapping, electrophoresis, SSCP, and RT-PCR.

5. The method of claim 1 wherein the mutation is detected in a protein.

6. The method of claim 1 wherein the mutant SQSTM1 protein is detected by a method selected from the group consisting of Western blot, immunoassay, ELISA, electrophoresis, anti-phospho amino acid antibodies, protein sequencing, proteolysis, functional assay, structure determination, and a measurement of size.

7. The method of claim 1 wherein the mutation is a type selected from the group consisting of missense, nonsense, deletion, insertion, intronic, silent, and splicing.

8. The method of claim 1 wherein the mutant SQSTM1 protein comprises a variant selected from the group consisting of A33V, V153I, P228L, V234V, K238del, H261H, S318P, R321C, S370P, P392L, G411S, and G425R.

9. The method of claim 1 wherein the mutation in the SQSTM1 gene comprises a mutation selected from the group consisting of c.98C>T, g.3′+7G>C, c.457G>A, g.5′-37C>T, c.683C>T, c.702G>A, c.714-716delGAA, c.783C>T, c.952T>C, c.961C>T, c.1108T>C, c.1175C>T, c.1231G>A, and c.1273G>A.

10. The method of claim 1 wherein the mutation produces a mutant protein comprising a change in a conserved region.

11. The method of claim 1 wherein the mutation affects the phosphorylation of a protein.

12. The method of claim 1 wherein the mutation produces a mutant protein comprising a change in a domain selected from the group consisting of Src homology domain (SH2); ZZ-type zinc finger domain; tumor necrosis factor receptor-associated factor 6 (TRAF6) binding site; a domain enriched in proline, glutamate, serine, and threonine (PEST domain); and a ubiquitin-association domain (UBA).

13. The method of claim 1 wherein the subject has or is predisposed to have familial amyotrophic lateral sclerosis, sporadic amyotrophic lateral sclerosis, Paget disease of bone (PDB), or neurodegeneration.

14. The method of claim 1 wherein the mutation is associated with a biological abnormality in a biological process selected from the group consisting of protein aggregation, protein folding, protein degradation, protein phosphorylation, and ubiquitination.

15. A composition comprising a first detection reagent adapted to detect a known marker of ALS and a second detection reagent adapted to detect:
a) a mutation in a SQSTM1 gene; or
b) a mutant SQSTM1 protein.

16. The composition of claim 15 wherein the second detection reagent is a biological molecule selected from the group consisting of a probe, a primer, and an antibody.

17. The composition of claim 15 wherein the second detection reagent is selected from the group consisting of:
a) an antibody adapted to detect specifically a mutant SQSTM1 protein; and
b) an oligonucleotide adapted to detect specifically a mutation in a SQSTM1 gene.

18. The composition of claim 17 wherein the antibody specifically detects a mutant SQSTM1 protein comprising a variant selected from the group consisting of A33V, V153I, P228L, V234V, K238del, H261H, S318P, R321C, S370P, P392L, G411S, and G425R.

19. The composition of claim 17 wherein the oligonucleotide specifically detects a mutation in the SQSTM1 gene selected from the group consisting of c.98C>T, g.3′+7G>C, c.457G>A, g.5′-37C>T, c.683C>T, c.702G>A, c.714-716delGAA, c.783C>T, c.952T>C, c.961C>T, c.1108T>C, c.1175C>T, c.1231G>A, and c.1273G>A.

20. A kit comprising:
a) a composition according to claims 15-19; and
b) an instruction for use.


SEQUENCE LISTING (7 sequences)

SEQ ID NO: 1 - protein, 65 aa, Homo sapiens
EPEAEAELCSVCEGASGPSEDNFLKNVGALESEGRPEELDPSQEEADPRLIDEGGWLTYDIGAAL

SEQ ID NO: 2 - protein, 65 aa, Pan troglodytes; identical to SEQ ID NO: 1

SEQ ID NO: 3 - protein, 65 aa, Bos Taurus
EPEAEAELCSACEGASGPSEDNFLKNVGALESGGQHEELDPSQEEADPRLIDEGGWLTYDIGAAL

SEQ ID NO: 4 - protein, 65 aa, Mus musculus
EPEAEAQLCSVCEGASAPPEDNFLKNVGALESVGQPEELDPSQEEADPRLIDEGGWLTYDIGAAL

SEQ ID NO: 5 - protein, 63 aa, Rattus norvegicus
EPEAELCSVCEGASAPSEDNFLKNVGALESVGQPEELDPSQEEADPRLIDEGGWLTYDIGAAL

SEQ ID NO: 6 - protein, 62 aa, Gallus gallus
LCSTCEGTNSQPQDTFLKNVGVIDPVPTGSLQSQELDVAQEEADPRLIDEGGWLTCDIGAAL

SEQ ID NO: 7 - protein, 52 aa, Danio rerio
LCPTCQSATASSQEYLKNIGSVSELNTASGTDADPRLVDEGGWLTYDIGGAL